(19) United States
(12) Patent Application Publication
(10) Pub. No.: US 2007/0044792 A1
(43) Pub. Date: Mar. 1, 2007

(54) Aerosol generators with enhanced corrosion resistance

(75) Inventor: Yehuda Ivri, Newport Beach, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AEROGEN, INC., Mountain View, CA (US)

(21) Appl. No.: 11/215,886

(22) Filed: Aug. 30, 2005

(51) Int. Cl. A61M 11/00

(52) U.S. Cl. 128/200.14

(57) ABSTRACT

An apparatus for generating an aerosol that includes a support member having a first face and a second face and defining an opening therethrough, where the support member includes a first material having a first galvanic potential, and an aerosolization element mounted on the support member and disposed substantially over the opening and defining at least one aperture therethrough. The aerosolization element may include a second material having a second galvanic potential that is substantially equal to the first galvanic potential of the first material. Also, a method of aerosolizing a liquid medicament by providing an aperture plate made of a first material and having a top surface and a bottom surface. The aperture plate is mounted on a support member that includes a second material that has an opening such that the aperture plate covers the opening, and where the first and second materials have similar galvanic potentials.

### CROSS-REFERENCES TO RELATED APPLICATIONS

This application is related to U.S. patent application Ser. No. 11/097,488, filed Apr. 1, 2005, which is a continuation of U.S. Pat. No. 6,915,962, filed May 20, 2003, which claims priority to U.S. Provisional Pat. App. No. 60/382,256, filed May 20, 2002. This application is also related to U.S. patent application Ser. No. 09/822,573, filed Mar. 30, 2001, and U.S. Pat. No. 6,755,189, filed May 18, 1999, and issued Jun. 29, 2004. The entire disclosures of all the above listed applications are herein incorporated by reference for all purposes.

### BACKGROUND OF THE INVENTION

This invention relates generally to the field of liquid dispensing, and in particular to the aerosolizing of fine liquid droplets. More specifically, the invention relates to aerosolization apparatuses having components designed to reduce galvanic corrosion when employed to produce such fine liquid droplets.

A great need exists for the production of fine liquid droplets. For example, fine liquid droplets are used in for drug delivery, insecticide delivery, deodorization, paint applications, fuel injectors, and the like. In many applications, it may be desirable to produce liquid droplets that have an average size down to about 0.5 μl. For example, in many medical applications, such a size is needed to insure that the inhaled drug reaches the deep lung.

Techniques for aerosolizing liquids are described in U.S. Pat. No. 5,261,601 and utilizes a perforate membrane disposed over a chamber. The perforate membrane comprises an electroformed metal sheet using a “photographic process” that produces apertures with a cylindrical exit opening.

Apparatuses and methods for producing fine liquid droplets also include those described U.S. Pat. Nos. 5,164,740; 5,586,550; and 5,758,637, the complete disclosures of which are herein incorporated by reference, describe exemplary devices for producing fine liquid droplets. These patents describe the use of aperture plates having tapered apertures to which a liquid is supplied. The aperture plates are then vibrated so that liquid entering the larger opening of each aperture is dispensed through the small opening of each aperture to produce the liquid droplets. Such devices have proven to be tremendously successful in producing liquid droplets.

Unfortunately many useful aerosolized compounds are made from corrosive liquid precursors. Solutions of liquid medicaments such as albuterol sulfate have low pHs (e.g., a pH of about 3.5 or less), which can quickly corrode the components of a aerosolizing apparatus that contains an aperture plate. Thus there is a need for aerosolization apparatuses that can operate for extended periods of time aerosolizing low pH, and other types of corrosive liquids. These and other issues are addressed by the present invention.

### BRIEF SUMMARY OF THE INVENTION

Embodiments of the invention include an apparatus for generating an aerosol. The apparatus may include a support member having a first face and a second face and defining an opening therethrough, where the support member includes a first material having a first galvanic potential. The apparatus may also include an aerosolization element mounted on the support member and disposed substantially over the opening, where the aerosolization element defines at least one aperture therethrough, and where the aerosolization element comprises a second material having a second galvanic potential that is substantially equal to the first galvanic potential of the first material. In some embodiments, the apparatus may include a vibratory element in mechanical communication with the support member, and a sealing member configured to isolate the vibratory element from a surrounding environment. The vibratory element may be operated to vibrate to cause movement of the aerosolization element in such a manner that a liquid at a first face of the aerosolization element can be dispensed as an aerosol through the at least one aperture. The sealing member may be made from an elastomer.

Embodiments of the invention also include an apparatus for generating an aerosol that includes a support member having a first face and a second face and defining an opening therethrough, where the support member comprises a first material having a first galvanic potential. The apparatus may also include an aerosolization element mounted on the support member and disposed substantially over the opening, where the aerosolization element defines at least one aperture therethrough, and where the aerosolization element includes a second material having a second galvanic potential that is substantially equal to the first galvanic potential of the first material. The apparatus may still further include a vibratory element in mechanical communication with the support member, where the vibratory element may be operated to vibrate to cause movement of the aerosolization element in such a manner that a liquid at a first face of the aerosolization element can be dispensed as an aerosol through the at least one aperture.

Embodiments of the invention still further include a method of aerosolizing a liquid medicament. The method may include the step of providing an aperture plate made of a first material and having a top surface and a bottom surface, and also having a plurality of apertures, where the aperture plate is mounted on a support member comprising a second material that has an opening such that the aperture plate covers the opening, and where the first and second materials have similar galvanic potentials. The method may also include supplying the liquid medicament to the bottom surface of the aperture plate, and vibrating the aperture plate to eject liquid droplets from the top surface of the aperture plate.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

A further understanding of the nature and advantages of the present invention may be realized by reference to the figures which are described in remaining portions of the specification. In the figures, like reference numerals are used throughout several to refer to similar components. In some instances, a sub-label consisting of a lower case letter is associated with a reference numeral to denote one of multiple similar components. When reference is made to a reference numeral without specification to an existing sub-label, it is intended to refer to all such multiple similar components.

FIG. 1 is a plan view of an aerosol generator assembly, in accordance with various embodiments of the present invention.

FIG. 2 a side cross-sectional view of an aerosol generator assembly, in accordance with various embodiments of the present invention.

FIG. 3 is a bottom view of an aerosol generator assembly, in accordance with various embodiments of the present invention.

FIG. 4 is a side view of an aerosol generator assembly, in accordance with various embodiments of the present invention.

FIG. 5 is a top perspective view of an aerosol generator assembly, in accordance with various embodiments of the present invention.

FIG. 6 is a bottom perspective view of an aerosol generator assembly, in accordance with various embodiments of the present invention.

FIG. 7 illustrates an aerosol generator assembly in accordance with various embodiments of the invention.

FIGS. 8A and 8B illustrate a portion of an aerosol generator assembly with a plurality of layers of bonding materials, in accordance with various embodiments of the invention.

FIGS. 9A and 9B illustrate a portion of an aerosol generator assembly having an adhesive situated between a support member and a vibratory element, in accordance with various embodiments of the invention.

FIG. 10 illustrates a cross section of an aerosol generator assembly having a single layer of bonding material applied to portions of a vibratory element, in accordance with various embodiments of the invention.

FIG. 11 illustrates a cross section of an aerosol generator assembly having a single layer of bonding material applied to portions of a vibratory element, a support member and an aerosolization element, in accordance with various embodiments of the invention.

FIG. 12 illustrates a cross section of an aerosol generator assembly having a first layer of bonding material applied to portions of a vibratory element and a second layer of bonding material applied to portions of the vibratory element, a support member and an aerosolizing element, in accordance with various embodiments of the invention.

FIG. 13 illustrates a cross section of an aerosol generator assembly having an adhesive disposed between a vibratory element and a support member, in accordance with various embodiments of the invention.

FIG. 14 illustrates a cross section of an aerosol generator assembly having an adhesive disposed between a vibratory element and a support member, a first layer of bonding material applied to portions of the vibratory element, and a second layer of bonding material applied to portions of the vibratory element, the support member, and an aerosolizing element, in accordance with various embodiments of the invention.

FIG. 15 illustrates a cross section of an aerosol generator assembly having an adhesive disposed between a vibratory element and a support member and first layer of bonding material applied to portions of the vibratory element, in accordance with various embodiments of the invention.

FIG. 16 illustrates a cross section of an aerosol generator assembly having an adhesive disposed between a vibratory element and a support member and first layer of bonding material applied to portions of the vibratory element, the support member, and an aerosolization element, in accordance with various embodiments of the invention.

FIG. 17 illustrates a cross section of an aerosol generator disposed within a mold assembly, in accordance with embodiments of the invention.

FIG. 18 illustrates a detail view of the aerosol generator and mold assembly of FIG. 17.

FIG. 19 illustrates a process flow diagram for producing an aerosol generator assembly in accordance with various embodiments of the invention.

### DETAILED DESCRIPTION OF THE INVENTION

The invention relates to methods and apparatuses for generating aerosols that include nebulizer components with improved resistance to galvanic corrosion. Galvanic corrosion normally increases with an increasing difference in the galvanic potential (e.g., oxidation potential) between two solid materials that come in electrical contact through a strong electrolytic solution (e.g., a strongly acidic solution). Making a support member component and aerosolization element of the nebulizer at least in part from materials having similar (or identical) galvanic potentials reduces the level of galvanic corrosion in these components as they aerosolize acidic solutions of medicaments (e.g., medicaments dissolved in solutions of HCl and H₂SO₄).

For example, an embodiment of the aerosolization apparatus may include an aperture plate made from palladium-nickel alloy that is mounted on a steel washer that has been made from, or coated with, the same palladium-nickel alloy. When these components come in contact with an acidic liquid medicament, galvanic corrosion is minimized because the exposed areas of both components are made from materials having the same galvanic potential. In contrast, when a palladium-nickel aperture plate is mounted on an uncoated steel washer, the difference in galvanic potentials causes the washer to act like an anode (i.e., the steel is oxidized), and the aperture plate to act like a cathode, of an electrolytic cell. The oxidation and corrosion of the steel washer in the medicament solution will interfere with, and eventually stop the operation of the apparatus.

Embodiments of the aerosolizaiton apparatus may include a support member having a first face and a second face and defining an opening therethrough that is made from (or coated with) a first material with a first galvanic potential, and an aerosolization element, made or coated with the same material, or another material having a similar galvanic potential. The aerosolization element may be mounted on the support member and disposed substantially over the opening, wherein the aerosolization element defines at least one aperture therethrough.

The apparatus may also include a vibratory element in mechanical communication with the support member, and a sealing member configured to isolate the vibratory element from a surrounding environment. The vibratory element may be operated to vibrate to cause movement of the aerosolization element in such a manner that a liquid at the first face of the aerosolization element can be dispensed as an aerosol through the at least one aperture. Some embodiments feature an electrode coupled to the vibratory element.

In accordance with certain embodiments, the vibratory element is annular in shape and/or comprises a piezoelectric ceramic. In other embodiments, the sealing element is annular, such that it covers at least a portion of the first face of the support member, at least a portion of the second face of the support member, and at least a portion of the vibratory element. In further embodiments, the sealing element can comprise an elastomer and/or a rubber, which can be, merely by way of example, a synthetic rubber or a silicone. The sealing member can be molded around at least a portion of the vibratory element, and can be formed by injection molding.

Some embodiments include one or more layers of bonding material between the sealing element and the vibratory element. The bonding material can couple the sealing member relatively securely to at least one of the vibratory element and the support member and can, in some cases, provide a relatively impervious barrier between the electrode and the surrounding environment. The bonding material can be relatively impervious to a relatively severe environmental condition, which can include, inter alia, heat, humidity, pressure, alternating cycles of vacuum and pressure, and a corrosive chemical.

In other embodiments, the bonding material can be selected from the group consisting of a paint, an epoxy, an adhesive and a primer, and the at least one layer of bonding material can comprise a first application of a first bonding material and a second application of a second bonding material. The first and second bonding materials can be the same bonding material. In further embodiments, the bonding material comprises an adhesive situated between the vibratory element and the support member. In some cases, the vibratory element comprises an inner surface and an outer surface, and the adhesive can be situated between the sealing element and at least a portion of at least one of the inner and outer surfaces of the vibratory element.

The present invention also relates to methods of making and/or using aerosol generator assemblies according to embodiments of the invention. One exemplary method for making an aerosol generator assembly comprises providing an aerosol generator, which can be similar to one of the aerosol generators discussed above. The method also includes providing a mold assembly formed to receive the aerosol generator, placing a mold material into the mold assembly, allowing mold material to form a sealing element about at least a portion of the aerosol generator and removing the aerosol generator from the mold assembly.

In some cases, placing the mold material into the mold assembly comprises injection molding the mold material. In other cases, the method includes preparing at least one of the vibratory element and the support member. Preparing the vibratory element and/or support member can comprise chemical etching of those components.

In certain embodiments, the method further comprises applying at least one layer of bonding material between the mold material and least one of the vibratory element and the support member. Applying at least one layer can include applying a layer of a first bonding material and applying a layer of a second bonding material. The first bonding material and the second bonding material can be the same bonding materials. One or more layers can be applied at room temperature and cured at a relatively high temperature for a specified period of time. The relatively high temperature is above about 100° C., more specifically between about 100° C. and about 150° C. In some cases, the relatively high temperature is between about 120° C. and about 140° C., and more specifically, about 130° C. In other cases, the specified period of time is between about 15 minutes and about 45 minutes.

Embodiments of the present invention include apparatus and methods for aerosolizing liquid. In accordance with one embodiment, an aerosol generator assembly is provided, comprising an aerosol generator and a sealing element overmolded onto the aerosol generator. Those skilled in the art will appreciate that, in accordance with certain embodiments of the invention, an aerosol generator comprises a piezoelectric and/or piezomagnetic vibratory element (a “piezo”) for vibrating an aerosolization element to aerosolize a fluid. In many cases, the piezoelectric member is driven by application of an electric and/or magnetic field, which often is supplied through an electric circuitry coupled to the piezo by one or more electrodes. The connection between the circuitry and the electrodes can be of any type that is operative to supply electric current to the piezo, including, for instance, conductive metal wires (optionally, with non-conductive insulation), conductive polymeric materials, and the like.

In accordance with some embodiments of the invention, a sealing member, which can comprise a variety of relatively impermeable and/or elastic substances (including, merely by way of example, elastomers, rubbers (both natural and synthetic), urethanes, silicon and the like) and can serve to isolate/protect the piezo and/or electrodes from the surrounding environment, which can sometimes include relatively severe environmental conditions, including without limitation, the conditions described below, such as relatively high heat, pressure, and atmospheric moisture, immersion in fluids, exposure to corrosive fluids, and the like.

Merely by way of example, a sealing member in accordance with some embodiments comprises a thermoplastic elsastomer known in the art as Santoprene™, which is commercially available from Advanced Elastomer Systems, L.P., of Akron, Ohio, USA. As described below, the sealing member can be formed by a variety of techniques, including for example, injection molding. U.S. Pat. No. 6,554,201, the entire disclosure of which is incorporated herein by reference for all purposes, describes one exemplary injection molding process that can be used in conjunction with aerosol generators.

In other embodiments, the sealing member can be used as a mounting apparatus for coupling the aerosol generator to a housing. Those skilled in the art will recognize that aerosol generators often are mounted within a housing for operation, such that the housing can provide (and/or be in communication with) a supply of fluid to be aerosolized, such as a chamber and/or the like. Additionally, the housing can also be an integrated part of a nebulizer system, such that it provides fluid communication between the aerosol generator and a patient's airway, either passively (such as, for instance, in an inhaler, where the patient inhales the aerosolized fluid from the housing) and/or actively (such as, for instance, when the housing is part of a respirator system). In some embodiments, therefore, the sealing member, which, as noted, can comprise a relatively elastic and/or flexible substance, can couple the aerosol generator to the housing securely enough to prevent dislodging of the generator, yet flexibly enough that the vibratory characteristics of the generator are not significantly impacted, thereby substantially maintaining the performance of the aerosol generator.

In still other embodiments, one or more bonding materials can be applied between and/or among the sealing member and various components of the aerosol generator. In some cases, bonding materials can include adhesives, epoxies, paints, primers and the like. Those skilled in the art will recognize that certain bonding materials can provide a relatively secure coupling between the aerosol generator and the overmold. Further, the bonding materials can be selected based on their abilities to enhance the vibratory performance of the generator and/or create or reinforce a barrier between the piezo (and/or its electrodes) and the surrounding environment. In many cases, the bonding materials are relatively impervious to environmental conditions to which aerosol generators commonly are exposed during operation, sanitization, etc. For instance, as discussed below, certain bonding materials can be relatively immune to an autoclave environment, which can introduce significantly elevated heat and pressure, along with relatively high levels of atmospheric water vapor and/or other fluids. Likewise, certain bonding materials can be impervious to any corrosive effects of cleaning fluids and/or fluids to be aerosolized.

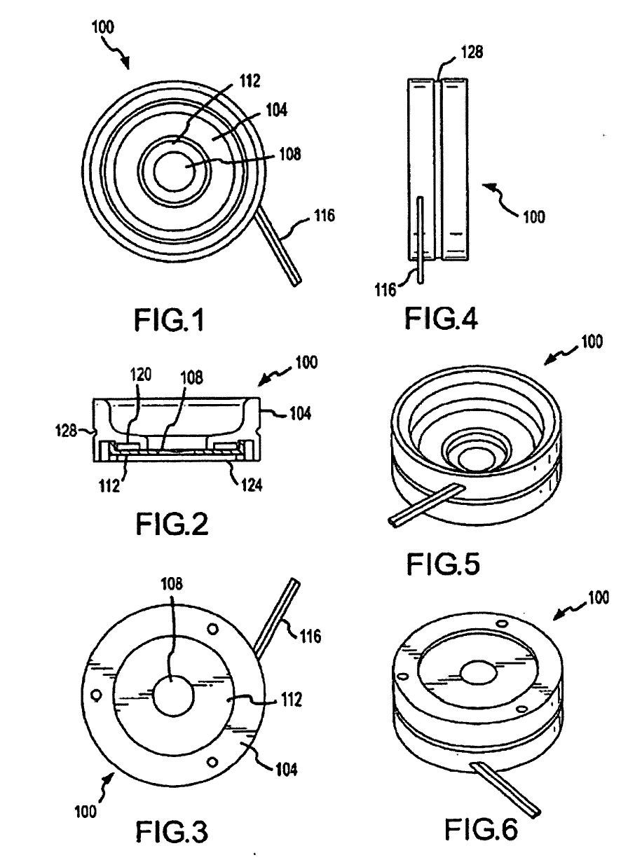

Turning now to FIG. 1, a top view of an aerosol generator assembly 100, including a sealing member 104, is illustrated, in accordance with certain embodiments of the invention. The aerosol generator assembly 100 further includes an aerosolization element 108 that includes a corrosion resistant material, a support member 112 that may include the same material, or another material that has a similar galvanic potential, and one or more electrical conduits 116. As illustrated by FIG. 2, a cross-sectional diagram of the assembly 100, an aerosol generator can further include a piezoelectric member 120, as well as a bottom plate 124. Although not apparent in the cross-sectional illustration of FIG. 2, those skilled in the art will appreciate from the view of FIG. 1 that the support member 112 can be annular in shape, thereby describing a central aperture, with the aerosolization element 108 bonded to the inner portion of the annular support member 112 and spanning the central aperture. Likewise, the piezo 120 can be annular in shape and can be bonded to a central and/or outer portion of the support member 112.

Also as illustrated by FIG. 2, the sealing member 104 can be formed in such a fashion as substantially to surround the piezo 120 and support member 112, and can, as illustrated in FIG. 1, be cup-shaped and/or annular in shape as well. Thus, in some embodiments, the sealing member 104 can be formed to have a relatively thick exterior portion that tapers to a relatively narrow interior portion, which can allow for more secure mounting in a housing without impacting the ability of aerosolized liquid to disperse away from the aerosolization element 108. To further facilitate mounting, the sealing member 104 can include one or more features (which may be integrally formed with the sealing member 104) to allow efficient coupling of the assembly 100 with the housing. Merely by way of example, the sealing member 104 of FIG. 2 includes a notch 128, which can be used for this purpose, in its exterior circumference.

FIG. 3 illustrates a bottom view of the aerosol generator assembly 100. As illustrated by FIG. 3, in accordance with certain embodiments, the sealing member 104 can extend around the outer surface of the generator to encompass a portion of the bottom face of the aerosol generator. In some cases, a portion of bottom face of the support member 112 may be left exposed, while in other cases, the sealing member 104 may extend inward across the bottom of the generator toward the support member's central aperture, leaving only the aperture plate 108 exposed. Also as shown on FIG. 3, the sealing member 104 may be formed to allow insertion of one or more electrical conduits 116 (e.g., insulated wires, etc.) through the sealing member 104 for coupling to a piezo, one or more electrodes, etc. In alternative embodiments, the electrical conduits 116 can be attached to the aerosol generator before formation of the sealing member 104, such that the sealing member 104 is molded around the conduits 132.

FIG. 4 illustrates a side view of the aerosol generator assembly 100, displaying the circumferential notch 128 described above, as well as the electrical conduits 132. FIGS. 5 and 6 illustrate perspective drawings of the assembly 100, as seen from the top and bottom, respectively.

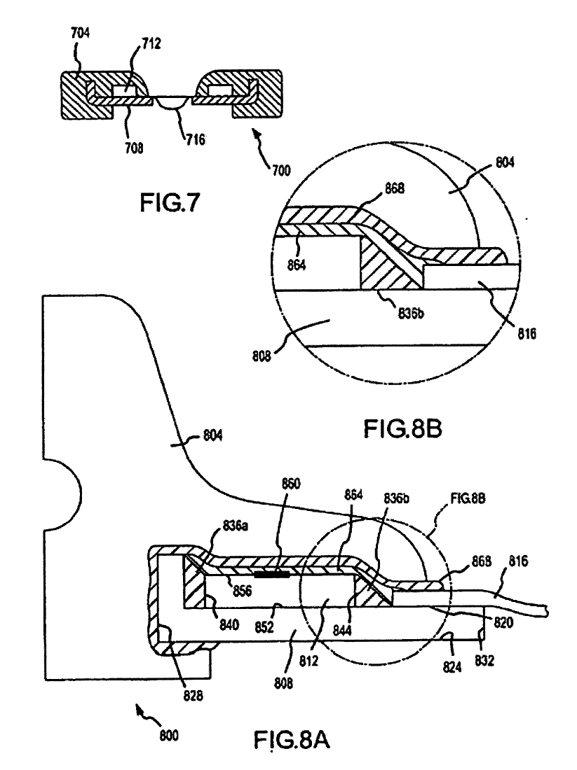

FIG. 7 provides a cross-sectional illustration of an aerosol generator assembly 700 in accordance with other embodiments of the invention. The aerosol generator assembly 700 includes a sealing member 704 formed around a support member 708 (e.g., an annular support member, such as a washer) that includes a corrosion resistant material and a vibratory element 712 in mechanical communication with one another. The assembly 700 further includes an aerosolization element 716 that includes the same corrosion resistant material as the support member 708 or another material that has a substantially equal galvanic potential, that is mounted on the support member 708 in a fashion similar to that described above. The sealing member 704 is generally annular in shape.

FIG. 8A illustrates a cross-sectional view of an aerosol generator assembly 800 in accordance with some embodiments of the invention. The assembly 800 features a sealing member 804 molded around an aerosol generator that includes a support member 808 made from (or coated with) a corrosion resistant material in mechanical communication with a piezoelectric vibratory element 812. An aerosolization element 816, which may also be made from (or coated with) the same corrosion resistant material, or a material having a similar galvanic potential, can be mounted on the support member 808 and can be used to aerosolize a liquid in a manner similar to that discussed above. The support member has a first face 820 and a second face 824. The support member 808 can be annular, having an outer surface 828 and an inner surface 832, which can define a central aperture through the aperture 808. In some cases, the outer surface 828 can define a flange. The aerosolization element 816 can be mounted so as to cover substantially the central aperture, and the aerosolization element 816 itself can have one or more apertures through which the aerosolized material can flow.

The vibratory element 812 can be in mechanical communication with the support member 808. For instance, the vibratory element 812 can be mechanically coupled to the support member 808 through a variety of means. Merely by way of example, the vibratory element 812 can be bonded to the support member 808 with an adhesive 836. For instance, in some cases, the vibratory element 812 may be attached with mechanical fasteners to the support member 808. In other cases, the vibratory element 812 and the support member 808 may be integrally formed, perhaps from the same material. In certain embodiments, as shown in FIG. 8A, the vibratory element 812 may be configured in a ring of rectangular cross-section, having an outer surface 840 and an inner surface 844, and the adhesive can be placed adjacent to either surface 840, 844, or both, to provide mechanical coupling between the vibratory element 812 and the support member 808. The vibratory element can also have a first face 852 and a second face 856, and one or more electrodes 860 may be mounted on either face 852, 856, or both. In some cases, a bonding material (for instance, an adhesive) may be placed between the first face 852 and the support member 808 and/or adjacent to the second face (either between the second face 856 and the electrodes 860 or over the second face and the electrodes 860, or both.

In some cases, one or more layers of bonding material may be disposed between and/or among the sealing member 804 and various components of the aerosol generator. As discussed above, one such bonding material may be an adhesive 844. Other bonding materials can include paints, epoxies, primers, and the like, as discussed herein. As illustrated by FIG. 8A, a first layer of bonding material 864 can be applied over the second face 856 of the vibratory element 812 and/or the electrode 860. The first layer 864 additionally can be disposed over any adhesive 836 adjacent to the outer 840 and/or inner 844 surfaces of the vibratory element. In some cases, as shown in FIGS. 8A and 8B, the adhesive 836 can be tapered, such that the first layer 864 can be applied to the point where the adhesive tapers to be flush with the support member 808, forming a barrier over the vibratory element 812, the electrode 860, and any adhesive 836. Depending on the embodiment, any of the bonding materials used herein can be applied as the first layer 864. In a particular embodiment, the first layer 864 can be paint and/or an epoxy.

Other embodiments can include one or more additional layers 868 of bonding material, which also can comprise any of the bonding materials discussed herein. The additional layers 868 can overlay the first layer 864 and can, additionally, be applied to portions of the support member 808. As illustrated more clearly by FIG. 8B, in some cases, the first layer 864 can be applied flush with (or slightly overlapping) the aerosolizing element 816, such that the additional layers 868 can be applied over the first layer and a portion of the aerosolizing element 816. If desired, the additional layers 868 can also be applied around the outer surface 828 of the support member and can cover at least a portion of the bottom face 824 of the support member.

In some embodiments, the sealing member 804 can comprise an elastomer, including any of those discussed above. In a particular embodiment, the sealing member 804 may comprise silicone. A silicone that may be used in accordance with the present invention is a two part silicone, available from a company known as Wacker-Chemie GmbH, Geschaftsbereich Silicone, Hanns-Seidel-Platz 4, D-81737 Muchen (Munich, Germany). Such silicone is known to be described in a product description captioned “Elastosil® LR 3003/10 A, B—LR 3003/80 A, B.” One variety of such silicone that may be used in accordance with the present invention is designated as 40 Shore, representative of relative hardness of the cured silicone.

As noted above, in some embodiments, prior to molding the sealing member 804 about the aerosol generator, a layer of primer (which can be thought of as one of the layers 868 in FIG. 8A) may be applied to surfaces of the aerosol generator receiving the overmold, which can include the vibratory element, the electrode, the portions of the support member, and a portion of the aerosolization element, such as the flange, or part of the flange. The primer may be applied to cover a slightly larger area than the overmold, to ensure that there is sufficient coverage of the primer to maximize bonding of the overmold. A primer that may be used in accordance with the present invention is known as CF6-135 High Technology Silicon Primer (Clear) available from a company known as NuSil Technology, Carpenteria, Calif., USA.

In other embodiments, a layer of epoxy (which can be thought of as the first layer 864 in FIG. 8A) may be applied to the exposed surfaces of the vibratory element. This layer can be applied prior to the application of a primer, or may be applied without the addition of a primer. The painted epoxy may comprise an autoclavable epoxy, such as, for example, a product designated as Masterbond EP3HTMED by a company known as Masterbond of New York, USA. The epoxy paint may be applied in a first layer and a second layer. In such case, it may be applied at room temperature with a fine point paintbrush. It may be cured at 130° C. for 30 minutes, whereupon a second application may be applied at room temperature, and likewise cured at 130° C. for 30 minutes.

It will be appreciated that the thickness of any bonding material (whether paint, primer, epoxy or the like) can be relatively small compared to the size of the aerosol generator and sealing member. As such, each bonding material may range from approximately a nanometer to approximately several micrometers in thickness, depending on the material used. Accordingly, in the various figures accompanying this application, the paint and primer thickness are enlarged for purposes of illustration.

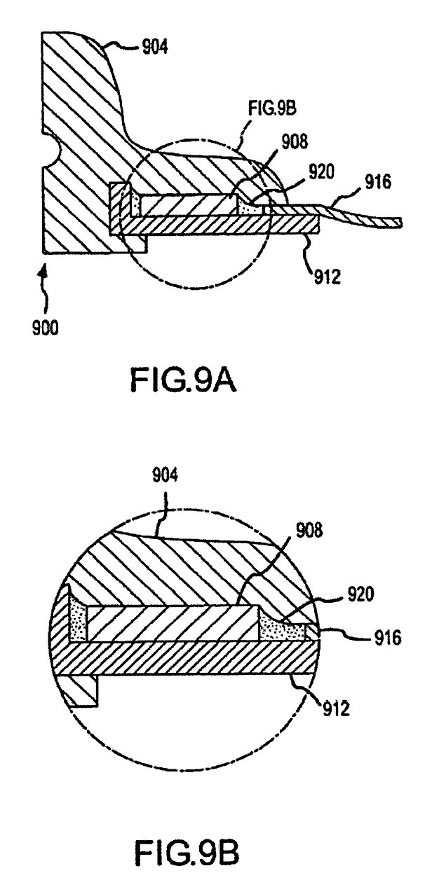

FIGS. 9A and 9B illustrate how an adhesive may be used in accordance with some embodiments of the invention. An aerosol generator assembly 900 may be formed of a sealing member 904 molded around an aerosol generator, which can comprise a vibratory element 908, a support member 912 and an aerosolizing element 916. A relatively thin layer of adhesive 920, which can be an epoxy adhesive, can be disposed between the vibratory element 908 and the support member 912. Excess adhesive may adhere to the sides of the vibratory element 908, and, in this way, can be used to provide a more secure fit between the sealing member 904 and the aerosol generator. Some embodiments, therefore, omit any additional bonding materials, as the adhesive 920 and sealing member act to provide a relatively impervious barrier between the surrounding environment and the vibratory element 908 (and, optionally, one or more electrodes, which are not shown in FIGS. 9A and 9B).

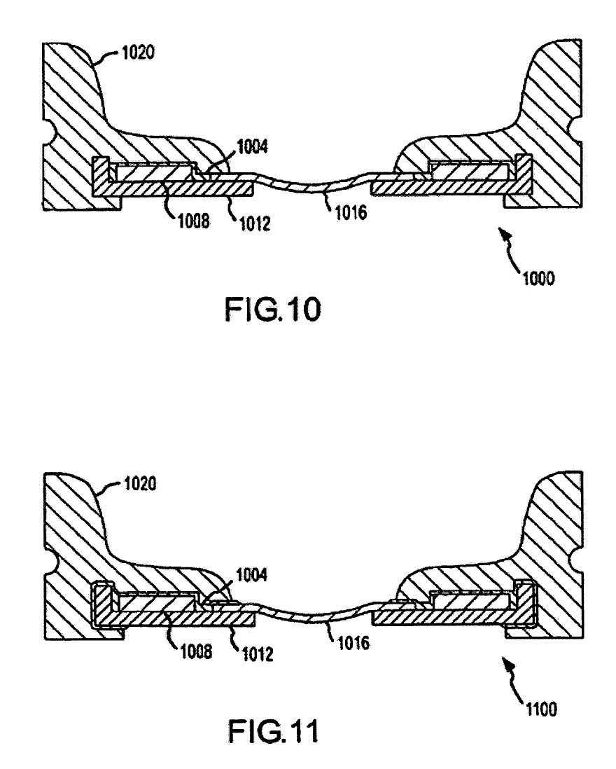

FIGS. 10-16 illustrate several different embodiments of the invention, employing a variety of bonding materials between and among the sealing member and various components of the aerosol generator. For ease of illustration, each of FIGS. 10-16 omit the electrodes, but those skilled in the art will appreciate, based on the disclosure herein, that electrodes could be incorporated as desired into each of the illustrated embodiments. Turning now to FIG. 10, an aerosol generator assembly 1000 is illustrated. The assembly includes a single layer 1004 of bonding material, which can be primer, paint, epoxy, etc., applied to the top face and each side face of a vibratory element 1008, which is mounted on a support member 1012. Hence, the bonding material 1004, in conjunction with the support member 1012, completely surrounds the vibratory element. In the assembly 1100 of FIG. 11, a layer of bonding material 1104 has been applied not only to surround the upper, inner and outer surfaces of the vibratory element 1008, but also has been applied to portions of the support member 1012 (including the flange and bottom surface thereof), as well as to portions of the aerosolizing element 1016. Thus, the bonding material 1104 has been applied to every surface of the aerosol generator with which the sealing member 1020 comes into contact.

The aerosolizing element 1016 may be an aperture plate constructed of a high strength and corrosion resistant material. As one example, the plate body may be constructed from a palladium nickel alloy (e.g., an alloy of about 80%, by wt., palladium and 20%, by wt. nickel). The element 1016 may also be made out of alloys comprising Ni—Cr—Mo, Ni—Cr—W, etc., which has a galvanic potential close to alloys of Pd—Ni. These materials are corrosion resistant to many corrosive materials particularly solutions with relatively low pH levels (e.g., pH of about 3.5 or less), such as medicament solutions for treating respiratory diseases by inhalation therapy, such as an albuterol sulfate and ipratroprium solution, which is used in many medical applications. Further, the palladium nickel alloy has a low modulus of elasticity and therefore a lower stress for a given oscillation amplitude. Other useful palladium nickel alloys are described generally in J. A. Abys, et al., “Annealing Behavior of Palladium-Nickel Alloy Electrodeposits,” Plating and Surface Finishing, August 1996, “PallaTech® Procedure for the Analysis of Additive IVS in PallaTech® Plating Solutions by HPLC” Technical Bulletin, Lucent Technologies, Oct. 1, 1996, and in U.S. Pat. No. 5,180,482, the complete disclosures of which are herein incorporated by reference. Materials in addition to Pd—Ni alloys that may be used to construct the aperture plate may also include other palladium alloys (e.g., Pd—Co alloys), and nickel alloys (e.g., Ni—Au alloys), gold, and gold alloys, as well as those described in U.S. Pat. No. 6,755,189, the entire contents of which are incorporated by this reference for all purposes.

The support member 1012 may be made or coated with a material that has a galvanic potential that is substantially equal to the galvanic potential of the aerosolizing element 1016. In some examples, the support member 1012 may be made from the same material as the aerosolizing element 1016, insuring the galvanic potentials for each component are equal. In other examples, the galvanic potentials are substantially equal enough so that the difference in the galvanic potentials is small than the difference between stainless steel and an alloy of 80% palladium and 20% nickel. In still other examples, the galvanic potentials are substantially equal enough to reduce the rate of galvanic corrosion of the material with lower galvanic potential below that observed when the support member 1012 is made of pure 316 stainless steel, and the aerosolizing element 1016 is made of a Pd—Ni alloy. Additional examples of materials that can be used in support member 1012 (as well as the aerosolizing element 1016) include alloys of nickel, chromium and other metals such as molybdenum and/or tungsten. For example, the alloy may include about 60% or more by weight of nickel and chromium, and/or about 80% or more by weight of nickel, chromium and molybdenum. Additional examples of the alloys include INCONEL® alloys from Haynes International of Kokomo, Ind., such as INCONEL® 625, which comprises an alloy of 21.5% by wt. Cr, 9% by wt. Mo, with the balance being Ni. Examples also include HASTELLOY® “C-Type” alloys from Haynes International of Kokomo, Ind., that have about 16-22% by wt. Cr, about 9-16% by wt. Mo, about 0-4% by wt. W, with the balance being Ni. HASTELLOY® C-276, for example includes about 16% by wt. chromium, about 16% by wt. molybdenum, about 4% by wt. tungsten, with the balance being nickel.

The support member 1012 and/or the aerosolizing element 1016 may also include alloys of cobalt, chromium, nickel, molybdenum and iron such as Conichrome®, Phynox™, and/or Elgiloy® produced by Fort Wayne Metals of Fort Wayne, Ind., among other alloys. In some formulations, Conichrome® may include about 40% by wt. cobalt, about 20% by wt. chromium, about 15% by weight nickel, about 7% by weight molybdenum; small amounts of carbon (e.g., about 0.06%), manganese (e.g., about 2%), silicon (e.g., about 0.5%), phosphorous (e.g., about 0.005%), sulfur (e.g., about 0.0015%), and beryllium (e.g., about 0.0002%); with the balance of the alloy including iron.

As noted above, achieving substantially equal galvanic potential between the support member 1012 and aerosolizing element 1016 may done by coating one or both components. For example, the support member 1012 may be machined from stainless steel and coated (e.g., electroplated, anodized, dipcoated, etc.) with a Pd—Ni alloy that is substantially equal to the galvanic potential of the aerosolizing element 1016.

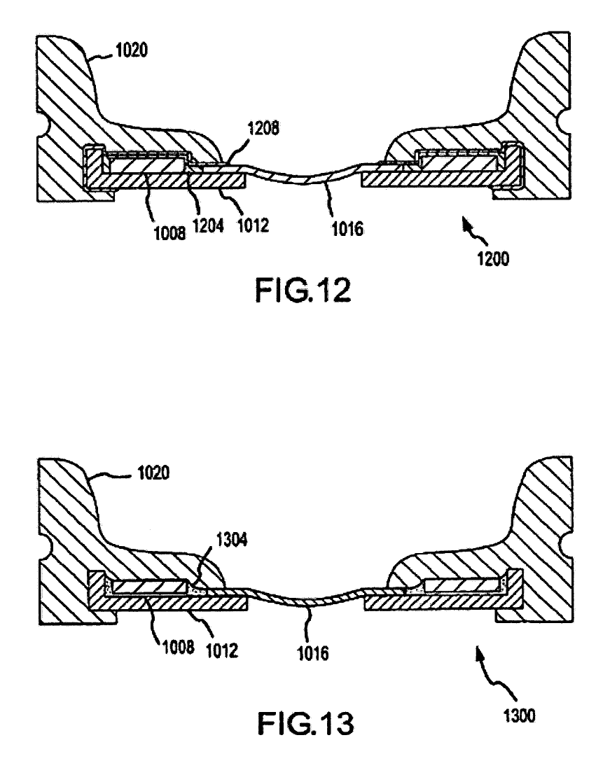

FIG. 12 illustrates an aerosol generator assembly 1200 employing a first layer 1204 and a second layer 1208 of bonding material. The first layer 1204 has been applied to the top, inner and outer surfaces of the vibratory element 1008, similar to layer 1004 in FIG. 10. The second layer 1208 is applied over the top of the first layer 1204 and also to portions of the support member 1216 (including, again, portions of the flange and bottom surfaces of the support member), as well as to the outer portion of the aerosolizing element 1016. Hence, like the layer 1104 in FIG. 11, the second layer 1208 has been applied to every surface of the aerosol generator with which the sealing member 1020 comes into contact.

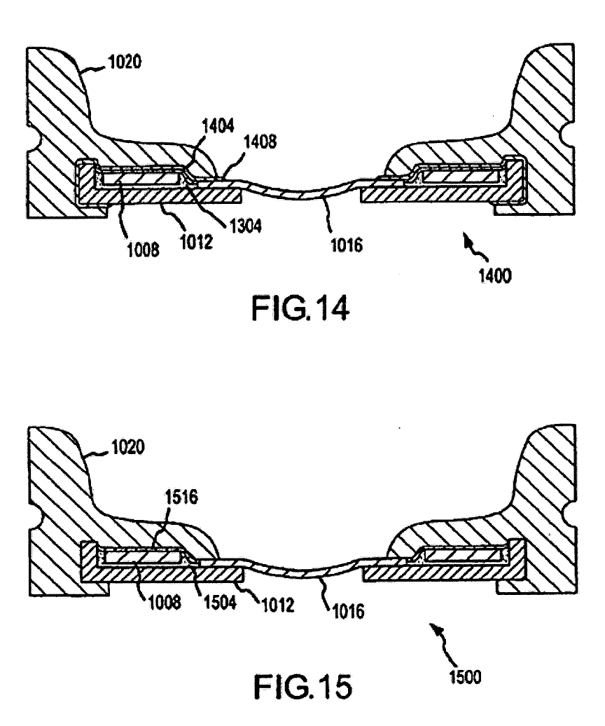

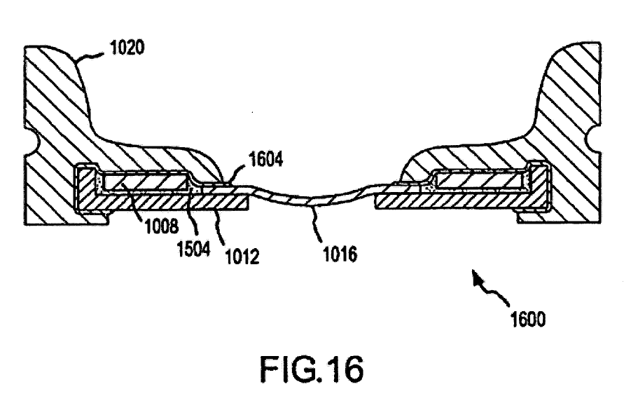

FIG. 13 illustrates an embodiment of an aerosol generator assembly 1300 similar to that discussed with respect to FIGS. 9A and 9B, in which the bonding material 1304 (perhaps an adhesive) is applied between the vibratory element 1008 and the support member 1012. In contrast, the assembly 1400 of FIG. 14 includes the adhesive 1304 between the vibratory element 1008 and the support member 1012, as well as additional layers 1404 and 1408, which can be thought of as similar to layers 1204, 1208 respectively, illustrated in FIG. 12. FIG. 15 illustrates an aerosol generator assembly 1500 in which a layer of adhesive 1504 is disposed between the vibratory element 1008 and the support member 1012. Further, a layer 1516 of bonding material overlays the adhesive 1504 and the vibratory element 1008, such that the adhesive 1504 and the layer 1516 of bonding material together function to completely encapsulate the vibratory element 1008. The assembly 1600 of FIG. 16 is similar to the assembly 1500 of FIG. 15, except that the layer of bonding material 1604 is applied not only to the adhesive 1504 and the vibratory element 1008, but also to portions of the support member 1012 and aerosolizing plate 1016, effectively coating each surface that will be in contact with the sealing member 1020.

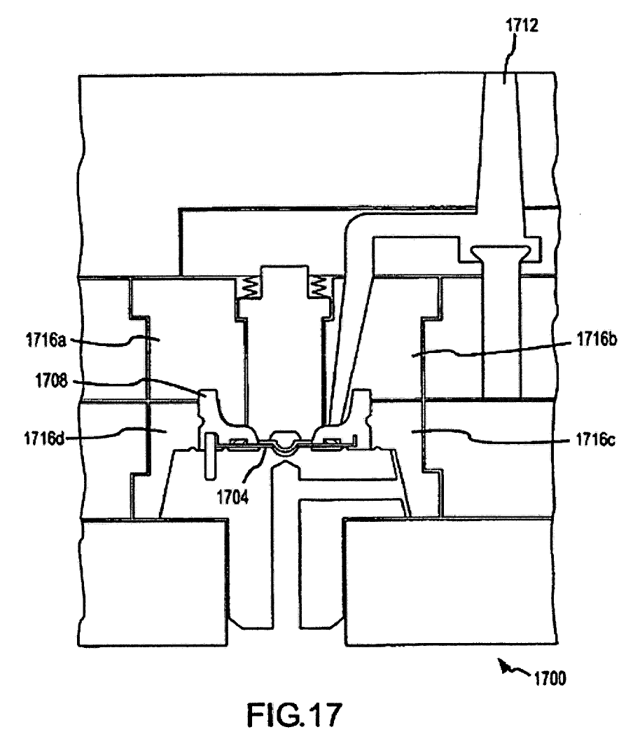

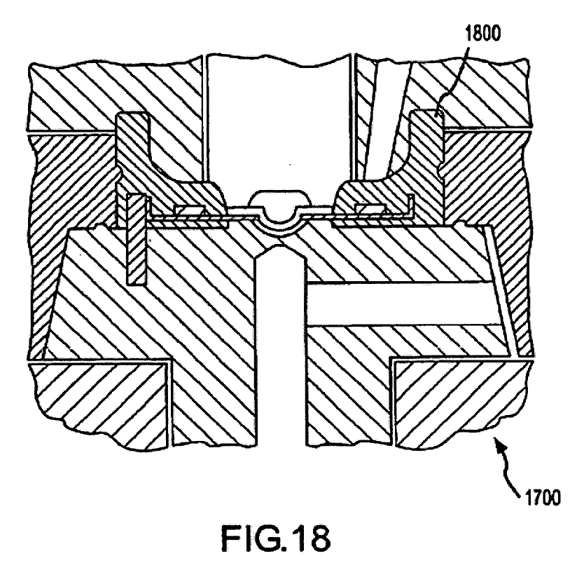

Turning now to the molding process, FIG. 17 illustrates an exemplary mold assembly 1700 for molding a sealing member onto an aerosol generator, in accordance with embodiments of the invention. The mold assembly 1700 is designed to accept an aerosol generator 1704 and defines a cavity 1708 into which mold material may be placed. The cavity defines the shape of the sealing member to be molded. In various embodiments, the mold material may comprise any of the materials discussed above with regards to the composition of a sealing member. In a particular embodiment, the mold material is capable of being injection molded. In other cases, the mold material can be in a liquid or semi-liquid form. The mold material can be placed into the cavity 1708 through any suitable method known in the art, including merely by way of example, injection molding via channel 1712. Those skilled in the art will appreciate that the mold assembly 1700 can comprise multiple components 1716a-d, which can be disassembled after the sealing member has hardened and/or cured, to allow for easy removal of the finished article. FIG. 18 illustrates a detail drawing of the mold assembly 1700 after mold material 1800 has been injected into the cavity.

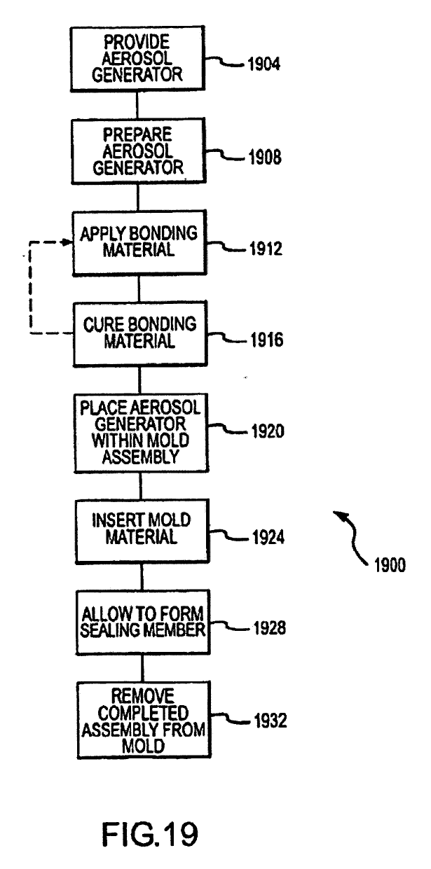

Hence, certain embodiments of the invention provide methods for creating aerosol generator assemblies. One exemplary embodiment 1900 is illustrated by FIG. 19. It should be noted that, while the procedures in method 1900 are illustrated and discussed in a certain order for ease of description, embodiments of the invention are not limited to any particular order.

The method 1900 comprises providing a aerosol generator (block 1904), which can, in some embodiments, include any of the aerosol generators discussed herein. At block 1908, the aerosol generator can be prepared to receive a bonding material and or to be molded with a sealing member. Preparation can include, inter alia, priming, scoring, chemical etching, and the like. At block 1912, a layer of bonding material, such as adhesive, epoxy, paint, primer and/or the like can be applied, and at block 1916 that layer can be cured. In some cases, the application of the bonding material can be done by dipping, paintbrush, airbrush, and/or other known application techniques. In other cases, the curing process can take place at a relatively high temperature, for a specified period of time. Optionally, the application (block 1912) and/or curing (block 1916) procedures can be repeated as necessary to produce multiple layers of bonding material and/or a single, thicker layer of material.

At block 1920, the aerosol generator can be placed within a mold assembly, and at block 1924, mold material may be placed into one or more appropriate cavities in the mold assembly. As noted above, block 1924 can include any appropriate procedure, including injection molding, packing, and the like. The mold material can then be allowed to form (e.g., cure, harden, etc.) to produce a sealing member molded onto the aerosol generator (block 1928), at which point the finished aerosol generator assembly can be removed from the mold assembly (block 1932).

Having described several embodiments, it will be recognized by those of skill in the art that various modifications, alternative constructions, and equivalents may be used without departing from the spirit of the invention. Additionally, a number of well known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the electrode“includes reference to one or more electrodes and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. An apparatus for generating an aerosol, comprising:
a support member having a first face and a second face and defining an opening therethrough, wherein the support member comprises a first material having a first galvanic potential; and
an aerosolization element mounted on the support member and disposed substantially over the opening, wherein the aerosolization element defines at least one aperture therethrough, and wherein the aerosolization element comprises a second material having a second galvanic potential that is substantially equal to the first galvanic potential of the first material.

2. The apparatus of claim 1, wherein the apparatus further comprises:
a vibratory element in mechanical communication with the support member; and
a sealing member configured to isolate the vibratory element from a surrounding environment;
wherein the vibratory element may be operated to vibrate to cause movement of the aerosolization element in such a manner that a liquid at a first face of the aerosolization element can be dispensed as an aerosol through the at least one aperture; and
wherein the sealing member comprises an elastomer.

3. The apparatus of claim 1, wherein the first and second materials are the same material.

4. The apparatus of claim 1, wherein the first material is an alloy of nickel and palladium.

5. The apparatus of claim 1, wherein the first material is an alloy comprising about 60% or more by weight of nickel and chromium.

6. The apparatus of claim 5, wherein the first material is an alloy comprising about 80% or more by weight of nickel, chromium, and molybdenum.

7. The apparatus of claim 5, wherein the first material comprises:
about 16% by wt. chromium;
about 16% by wt. molybdenum;
about 4% by wt. tungsten, and
about 64% by wt. nickel.

8. The apparatus of claim 1, wherein the second material is an alloy of nickel and palladium.

9. The apparatus of claim 1, wherein the second material is an alloy comprising about 60% or more by weight of nickel and chromium.

10. The apparatus of claim 9, wherein the second material is an alloy comprising about 80% or more by weight of nickel, chromium, and molybdenum.

11. The apparatus of claim 9, wherein the second material comprises:
about 16% by wt. chromium;
about 16% by wt. molybdenum;
about 4% by wt. tungsten, and
about 64% by wt. nickel.

12. The apparatus of claim 1, wherein the first material comprises an alloy of cobalt, chromium, nickel, molybdenum and iron.

13. The apparatus of claim 1, wherein the second material comprises an alloy of cobalt, chromium, nickel, molybdenum and iron.

14. The apparatus of claim 1, wherein the first material forms a coating over the surface of the support member.

15. The apparatus of claim 14, wherein an interior portion of the support member comprises a third material having a different galvanic potential than the first material.

16. The apparatus of claim 15, wherein the first material is an alloy of nickel and palladium and the third material is stainless steel.

17. The apparatus of claim 1, wherein the first material is gold, or an alloy of gold.

18. The apparatus of claim 1, wherein the second material is gold, or an alloy of gold.

19. An apparatus for generating an aerosol, the apparatus comprising:
a support member having a first face and a second face and defining an opening therethrough, wherein the support member comprises a first material having a first galvanic potential;
an aerosolization element mounted on the support member and disposed substantially over the opening, wherein the aerosolization element defines at least one aperture therethrough, and wherein the aerosolization element comprises a second material having a second galvanic potential that is substantially equal to the first galvanic potential of the first material; and
a vibratory element in mechanical communication with the support member, wherein the vibratory element may be operated to vibrate to cause movement of the aerosolization element in such a manner that a liquid at a first face of the aerosolization element can be dispensed as an aerosol through the at least one aperture.

20. The apparatus of claim 19, further comprising a sealing member configured to isolate the vibratory element from a surrounding environment.

21. The apparatus of claim 20, wherein the sealing member comprises an elastomer.

22. The apparatus of claim 19, wherein the first and second materials are the same material.

23. The apparatus of claim 19, wherein the first material is an alloy of nickel and palladium.

24. The apparatus of claim 19, wherein the first material is an alloy comprising about 60% or more by weight of nickel and chromium.

25. The apparatus of claim 24, wherein the first material is an alloy comprising about 80% or more by weight of nickel, chromium, and molybdenum.

26. The apparatus of claim 24, wherein the first material comprises:
about 16% by wt. chromium;
about 16% by wt. molybdenum;
about 4% by wt. tungsten, and
about 64% by wt. nickel.

27. The apparatus of claim 19, wherein the second material is an alloy of nickel and palladium.

28. The apparatus of claim 19, wherein the second material is an alloy comprising about 60% or more by weight of nickel and chromium.

29. The apparatus of claim 19, wherein the second material is an alloy comprising about 80% or more by weight of nickel, chromium, and molybdenum.

30. The apparatus of claim 19, wherein the second material comprises:
about 16% by wt. chromium;
about 16% by wt. molybdenum;
about 4% by wt. tungsten, and
about 64% by wt. nickel.

31. The apparatus of claim 19, wherein the first material comprises an alloy of cobalt, chromium, nickel, molybdenum and iron.

32. The apparatus of claim 19, wherein the second material comprises an alloy of cobalt, chromium, nickel, molybdenum and iron.

33. The apparatus of claim 19, wherein the first material forms a coating over the surface of the support member.

34. A method of aerosolizing a liquid medicament, the method comprising:
providing an aperture plate made of a first material and having a top surface and a bottom surface, and also having a plurality of apertures, wherein the aperture plate is mounted on a support member comprising a second material that has an opening such that the aperture plate covers the opening, and wherein the first and second materials have similar galvanic potentials;
supplying the liquid medicament to the bottom surface of the aperture plate; and
vibrating the aperture plate to eject liquid droplets from the top surface of the aperture plate.

35. The method of claim 34, wherein the first and second materials are the same material.

36. The method of claim 34, wherein the first and second materials are an alloy of palladium and nickel.

37. The apparatus of claim 34, wherein the first and second materials are an alloy comprising about 60% or more by weight of nickel and chromium.

38. The apparatus of claim 34, wherein the first and second materials are an alloy comprising about 80% or more by weight of nickel, chromium, and molybdenum.

39. The apparatus of claim 34, wherein the first and second materials comprise:
about 16% by wt. chromium;
about 16% by wt. molybdenum;
about 4% by wt. tungsten, and
about 64% by wt. nickel.

40. The method of claim 34, wherein the liquid medicament has a pH of about 3.5 or less.

41. The method of claim 34, wherein the liquid medicament comprises albuterol sulfate.

42. The method of claim 34, wherein the plurality of apertures in the aperture plate taper in a direction from the top surface to the bottom surface of the aperture plate.

43. The method of claim 42, wherein the plurality of apertures have an exit angle that is in a range from about 30° to about 60°, and a diameter in the range from about 1 μm to about 10 μm at the narrowest portion of the tapered aperture.
